(19) United States
(12) Patent Application Publication
(10) Pub. No.: US 2024/0285948 A1
(43) Pub. Date: Aug. 29, 2024

(54) CORRECTIVE SLEEP APNEA DEVICE

(71) Applicants: Yince Loh, Seattle, WA (US); Sukhdeep S. Dhaliwal, Bainbridge Island, WA (US)

(72) Inventors: Yince Loh, Seattle, WA (US); Sukhdeep S. Dhaliwal, Bainbridge Island, WA (US)

(21) Appl. No.: 18/683,729

(22) PCT Filed: Aug. 15, 2022
(86) PCT No.: PCT/US2022/074987

§ 371 Date: Feb. 14, 2024

Related U.S. Application Data
(60) Provisional application No. 63/233,637, filed on Aug. 16, 2021.

(51) Int. Cl. A61N 1/36; A61N 1/05

(52) U.S. Cl. CPC A61N 1/3611; A61N 1/05; A61N 1/36139

(57) ABSTRACT

A corrective sleep apnea device using physically separate actuator portions that active the actuator combination when in proximity one another, that is easy to use and does not require significant invasive surgery to be installed in the user or that may be, but is not limited to, a device that does not require a battery be implanted in the user and does not require further medical procedures in the future to replace an implanted battery.

### REFERENCE TO RELATED APPLICATION

This application claims priority to U.S. Provisional Patent Application Ser. No. 63/233,637 filed Aug. 16, 2021, the disclosure of which is incorporated herein in its entirety.

### BACKGROUND

Obstructive sleep apnea (“OSA”) is a condition marked by abnormal breathing during sleep. OSA occurs when the airway at the back of the throat becomes physically blocked by the tongue or other soft tissues in the oral and pharyngeal cavity. Because of this blockage of the throat, people with OSA have multiple extended pauses in breath during the night-time period when they try to sleep. These temporary breathing lapses cause lower-quality sleep and affect the body's supply of oxygen, leading to potentially serious health consequences. It independently contributes to stroke and cardiovascular disease and death. OSA affects nearly one billion people worldwide and is one of the most common sleep disorders in the United States.

The primary way to treat OSA is the use of continuous positive airway pressure (“CPAP”) devices. These CPAP devices use a pump to deliver a continuous flow of air via a sealed mask covering the nose and face which maintains a positive pressure setting. The generated continuous flow of air from these CPAP devices keeps the tongue, uvula and soft palate from shifting too far into the airway, maintaining patency of the airway. Despite the effectiveness of CPAP as a treatment for OSA, compliance remains low due to several factors related to the necessity of the mask/seal arrangement; the noisiness of the required pump; the continuous requirement to clean the equipment involved (e.g. the CPAP device tubing and mask); the discomfort of the mask, the humidification of the air delivered; and how the mask and tubing impair spontaneous movement and repositioning during sleep.

Because of the limitations of CPAP devices, alternatives have been developed. Some alternatives have included oral appliances, uvulopalatalpharyngoplasty (UPPP), and mandibular advancement. The latter two are invasive surgical procedures that carry a risk of procedural complications that may disable the patient. Another approach, which also requires a fairly invasive surgical procedure, utilizes an implanted battery attached to implanted leads that connect to the hypoglossal nerve of the tongue. With this device, when sleep obstruction is detected, the implanted leads stimulate the hypoglossal nerve, which causes the tongue to protrude forward, opening the airway and allowing the sleeping person to continue to breath relatively normally. Besides this approach requiring a fairly invasive surgery, since the battery is implanted in the person using this system, this person must at times go in for follow-up procedures to replace the implanted battery. An additional drawback is that some people do not tolerate the tongue protrusion or the nerve stimulation, as it does not selectively pull the base of the tongue forward. Furthermore, it has a narrow therapeutic window, as it needs to stimulate the nerve enough to move the tongue, yet not enough to wake the person from sleep. All of these approaches seek to widen the airway directly by, sometimes, advancing the tongue, which is held in tight conjunction with the mandible, towards the front of the mouth.

Accordingly, there is a need for a corrective sleep apnea device that addresses one or more of the limitations of past approaches, where the device may be, but is not limited to, a device that does not require an invasive surgery to be installed in the user or may be, but is not limited to, a device that does not require a battery be implanted in the user that requires further medical procedures in the future to replace the implanted battery.

### SUMMARY

According to one aspect of the present invention, a corrective sleep apnea device may include an actuator combination having a first portion and a second portion that are physically separate from each other, where the first portion is implanted into a head of a user; and the first portion and the second portion create an active state of the actuator combination when the first portion and the second portion are in proximity to one another and when in an active state, the actuator combination moves the tongue of the user forward, keeping the oral airway and posterior nasopharyngeal space of the user open while the user sleeps.

According to another aspect of the present invention, a corrective sleep apnea device may include an occlusal appliance and an actuator combination having a first magnetic portion and a second magnetic portion that are physically separate from each other, where the first magnetic portion is implanted into a tongue of a user; the second magnetic portion is housed in the occlusal appliance; and the first magnetic portion and the second magnetic portion create an active state of the actuator combination when the occlusal appliance is inserted into the mouth, putting the first magnetic portion and the second magnetic portion in proximity to one another and when in an active state, the actuator combination moves the tongue of the user forward, keeping the oral airway and posterior nasopharyngeal space of the user open while the user sleeps.

According to another aspect of the present invention, a corrective sleep apnea device may include an adhesive pad; and an actuator combination having a first micro-implantable pulse generator portion and a second controller portion that are physically separate from each other, where the first micro-implantable pulse generator portion is implanted into an oral muscle of a user; the second controller portion is housed in the adhesive pad; the first micro-implantable pulse generator portion and the second controller portion create an active state of the actuator combination when the adhesive pad is attached to the chin of the user, putting the first micro-implantable pulse generator portion and the second controller portion in proximity to one another; and when in an active state, the actuator combination stimulates the oral floor muscles of the user, causing the tongue of the user to move forward, keeping the oral airway and posterior nasopharyngeal space of the user open while the user sleeps.

### DRAWINGS

Objects, features, and advantages of the present invention will become apparent upon reading the following description in conjunction with the drawing figures, in which:

FIG. 1 is a perspective view of an embodiment of components of a corrective sleep apnea device of the present invention;

FIG. 2A is a front view of an occlusal appliance of an embodiment of the present invention;

FIG. 2B is a side view of an occlusal appliance of an embodiment of the present invention;

FIG. 2C is a plan view of an occlusal appliance of an embodiment of the present invention;

FIGS. 3A-3C depict an embodiment of a method for inserting a left side actuator of a first portion of an actuator combination of the present invention into a user's tongue;

FIG. 4 is a cross-sectional side view of the lower portion of a user of an embodiment of the present invention's head depicting a first portion of an actuator combination of the present invention implanted into a user's tongue with an embodiment of an occlusal appliance of the present invention prior to insertion into the user's mouth;

FIG. 5A is a cross-sectional side view of the lower portion of a user of an embodiment of the present invention's head depicting a first portion of an actuator combination of the present invention implanted into a user's tongue with an embodiment of an occlusal appliance of the present invention inserted into the user's mouth;

FIG. 5B is an assembly view with a first portion of an actuator combination of the present invention implanted into a user's tongue depicting an embodiment of an occlusal appliance of the present invention being engaged with the teeth of a user of the present invention;

FIG. 6 is a cross-sectional side view of the lower portion of a user of the present invention's head depicting another embodiment with a first portion of an actuator combination of the present invention implanted into a user's oral floor muscle with an embodiment of a second portion of an actuator combination of the present invention prior to attachment of the second portion of the actuator combination underneath the user's chin;

FIG. 7A is a cross-sectional side view of the lower portion of a user of the present invention's head depicting another embodiment with a first portion of an actuator combination of the present invention implanted into a user's oral floor muscle with an embodiment of a second portion of an actuator combination of the present invention with the second portion of the actuator combination attached underneath the user's chin and in an inactive state;

FIG. 7B is a cross-sectional side view of the lower portion of a user of the present invention's head depicting another embodiment with a first portion of an actuator combination of the present invention implanted into a user's oral floor muscle with an embodiment of a second portion of an actuator combination of the present invention with the second portion of the actuator combination attached underneath the user's chin and in an active state;

FIGS. 8A-8C depict an embodiment of another method for inserting a left side actuator of a first portion of an actuator combination of the present invention into a user's tongue;

FIG. 9 depicts an embodiment of another method for inserting a left side actuator of a first portion of an actuator combination of the present invention into a user's mouth; and

FIG. 10 is a cross-sectional side view of the lower portion of a user of an embodiment of the present invention's head depicting a first portion of an actuator combination of the present invention transvascularly implanted into a user's vein with an embodiment of an occlusal appliance of the present invention inserted into the user's mouth.

### DESCRIPTION

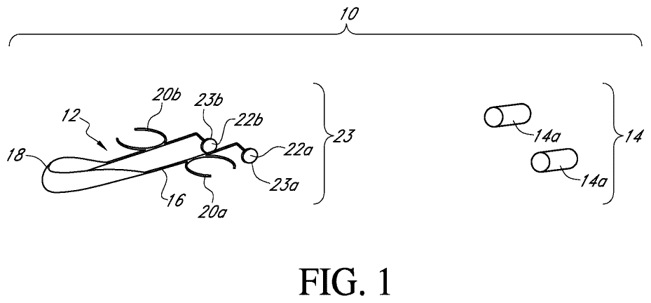

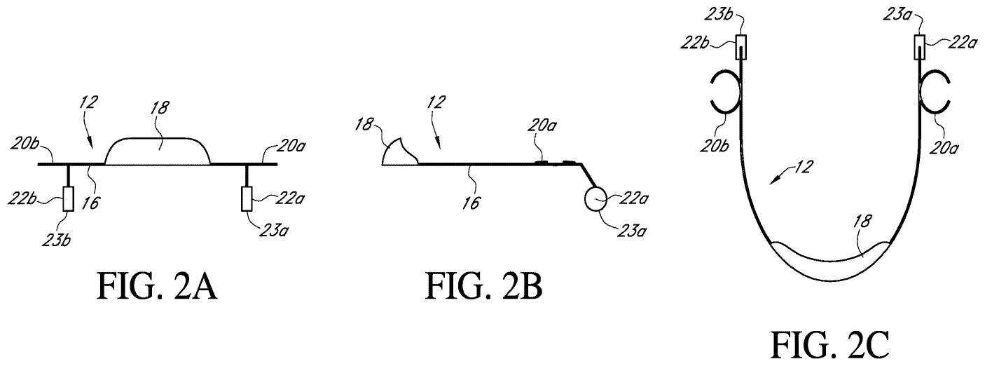

Referring to FIG. 1 and FIGS. 2A-2C, an embodiment of a corrective sleep apnea device 10 of the present invention is depicted. The corrective sleep apnea device 10 of this embodiment includes an occlusal appliance 12 and a first portion 14 of an actuator combination. In this embodiment, the first portion 14 of the actuator combination has two actuators 14a, 14b and each is preferably discoid or cylindrical in shape, 5 mm in diameter with a height of approximately 2-5 mm. It should be understood that in other embodiments the first portion 14 of the actuator combination can be a single actuator or more than two actuators. The occlusal appliance 12 of this embodiment is a retainer, which includes a frame 16, an inner arch 18, a pair of anchors 20a, 20b and a pair of winglets 22a, 22b which house a second portion 23 of the actuator combination. In this embodiment, the second portion 23 of the actuator combination has two actuators 23a, 23b. It should be understood that in other embodiments the second portion 23 of the actuator combination can be a single actuator or more than two actuators. The retainer 12 of this embodiment is similar to other orthodontic appliances, except for the inclusion of the winglets 22a, 22b. In this embodiment, the first portion 14a, 14b and the second portion 23a, 23b of the actuator combination are magnets of opposite polarity that attract to each other when in proximity to one another. In another embodiment, the first portion 14 of an actuator combination is a magnetic field generator using a microcontroller to switch between active and passive magnetic states.

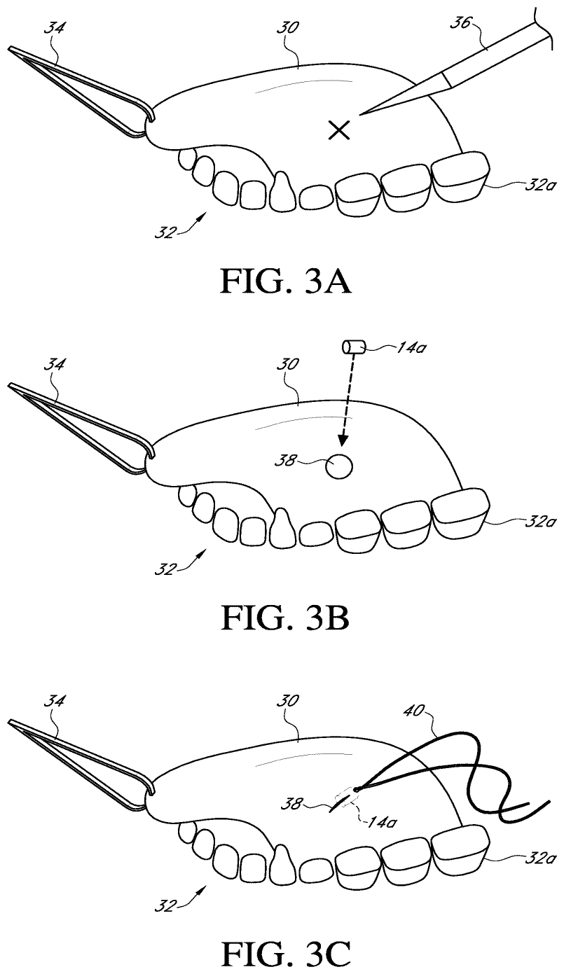

In one embodiment, to configure the corrective sleep apnea device 10 for use, the actuators 14a, 14b of the first portion 14 of the actuator combination are surgically implanted into the tongue 30 of a user, using minimally invasive techniques. Referring to FIGS. 3A-3C, one embodiment of a method for inserting the actuators 14a, 14b of the first portion 14 of the actuator combination into a user's tongue 30 is depicted. The tongue 30 and teeth 32 of the user of the invention are depicted in FIGS. 3A-3C. In this embodiment, referring first to FIG. 3A, the person inserting the left side actuator 14a of the first portion 14 of the actuator combination uses a puller 34 to distract and pull the tongue 30 of the user of the invention forward toward the front of the mouth and distracting the tongue 30 to the opposite side to maximize exposure to the target area. The actuators 14a, 14b of the first portion 14 of the actuator combination are positioned on a lateral aspect of the muscle at the junction of the posterior one-third and anterior two-thirds of the tongue 30. This junction can be identified by the row of Vallate papillae. With the tongue 30 in a forward and a laterally distracted position, the person inserting the left side actuator 14a of the first portion 14 of the actuator combination cuts an incision into the tongue 30 with a blade 36 using known surgical techniques in order to place the left side actuator 14a of the first portion 14 of the actuator combination in the vertical fibers of the intrinsic muscle of the tongue 30. After administration of local anesthetic with epinephrine, a 6 mm vertical incision is made just below the Filiform papillae through the mucosa. Next, blunt dissection is carried out to a depth of approximately 7 mm, creating a soft tissue pocket 38. In this embodiment, the pocket 38 is formed in a location such that when the left side actuator 14a of the first portion 14 of the actuator combination is implanted, it is at an optimal location to interact with the left side actuator 23a of the second portion 23 of the actuator combination housed in the winglets 22a, 22b when the retainer 12 is in the user's mouth 48. Referring to FIG. 3B, with the pocket 38 formed, the left side actuator 14a of the first portion 14 of the actuator combination is then placed into the pocket 38, aligned and then embedded into the pocket 38. After the left side actuator 14a of the first portion 14 of the actuator combination is implanted, as depicted in FIG. 3C, it is next fixated in position with 4-0 proline suture 40 and layered closure is performed with known surgical techniques. This same technique is repeated on the opposite side of the tongue 30 to implant the right side actuator 14b of the first portion 14 of the actuator combination.

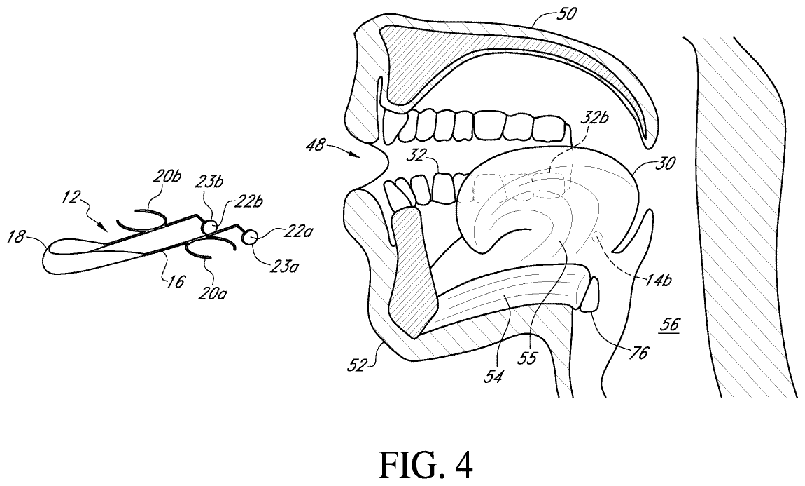

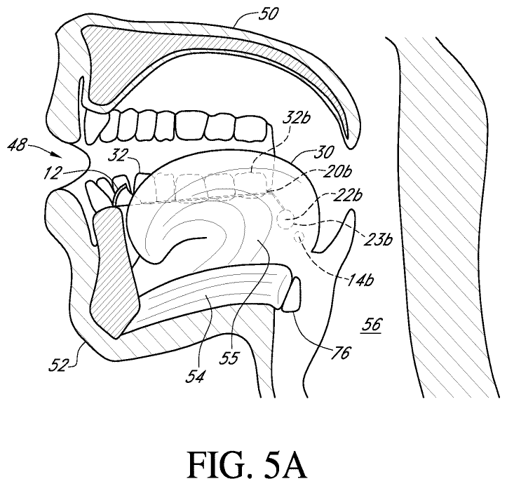

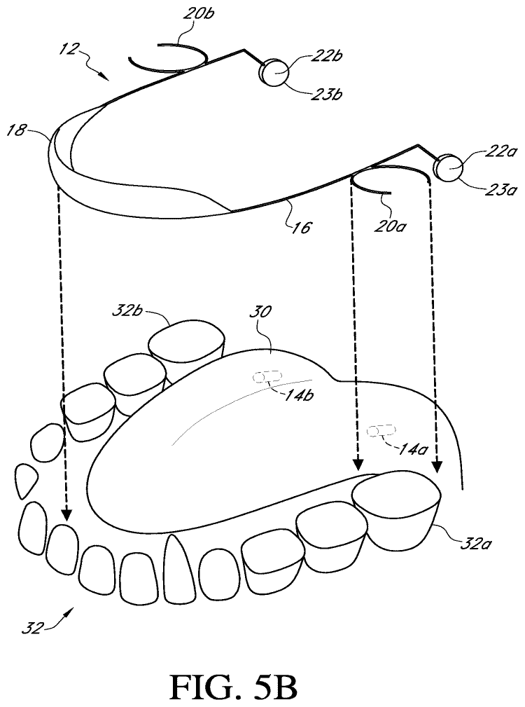

With first portion 14 of the actuator combination implanted in the tongue 30, the user is ready to use the corrective sleep apnea device 10. Referring to FIG. 4, a side view of the interior of the user's mouth 48 with a first portion 14 of the actuator combination implanted is shown. In addition to other elements, the user's upper jaw 50, lower jaw 52, geniohyoideus muscle 54, genioglossus muscle 55, tongue 30, teeth 32 and oral airway and posterior nasopharyngeal space 56 are depicted. The implanted first portion 14 of the actuator combination sits at the base of the tongue or oral floor 30, near the user's airway 56. In this FIG. 4, the user is about to go to sleep. In this view, the user has grasped the retainer 12 in his hand and is about to put the retainer 12 into his mouth 48. Referring now to FIGS. 5A, 5B, the user puts the retainer 12 into his mouth 48 between the upper jaw 50 and the lower jaw 52 and along the back of his teeth 32. The user places the anchors 20a, 20b around their respective molars 32a, 32b, which serve as the anchor teeth in the mouth 48 for the retainer 12. The retainer 12 is now in place, abutting the lower dental arch, and the position of the second portion 23 of the actuator combination is in a fixed position to the lower jaw 52. With the retainer 12 in this position, the second portion 23a, 23b of the actuator combination in the winglets 22a, 22b now interacts with the implanted first portion 14a, 14b of the actuator combination implanted in the tongue 30 to generate a force resulting in forward movement of the base of the tongue only, without unnecessarily contracting the entire tongue musculature and without any resultant tongue protrusion. This puts the corrective sleep apnea device 10 in an active state. This movement of the tongue 30 keeps the user's oral airway and posterior nasopharyngeal space 56 open while the user sleeps. Then in the morning, when the user is no longer sleeping, he removes the retainer 12 from his mouth 48, and the actuator portions 14, 23 are no longer in proximity, returning the actuator combination to an inactive state. The user, with the retainer 12 removed from his mouth 48, proceeds with his normal daily activities.

Furthermore, in some embodiments, the corrective sleep apnea device 10 may include one or more sensors or devices external to the mouth that collect physiological data associated with the corrective sleep apnea device 10 user's sleep functions. In these embodiments, these sensors or devices are typically non-invasive, such as wearable smart devices, and they are programmed to communicate with the second portion 23 of the actuator combination. In certain embodiments, these sensors or devices are programmed such that when predetermined physiological parametric thresholds are met, instructions are sent to the second portion 23 of the actuator combination to switch the device 10 to an active state. For example, in one embodiment, the second portion 23a, 23b of the actuator combination is a variable state electromagnetic field generator that receives instructions from such an external smart device or wearable device. In this embodiment, the smart device or wearable device is programmed with a sleep function threshold, and when that threshold is reached, the smart device or wearable device, triggers the second portion 23a, 23b of the actuator combination putting it in an active magnetic state which generates force resulting in forward movement of the base of the tongue only. It should be understood that in other embodiments, the first portion 14a, 14b of the actuator combination, instead of the second portion 23a, 23b, may be a variable state electromagnetic field generator or that both portions 14, 23 of the actuator combination may be variable state electromagnetic field generators.

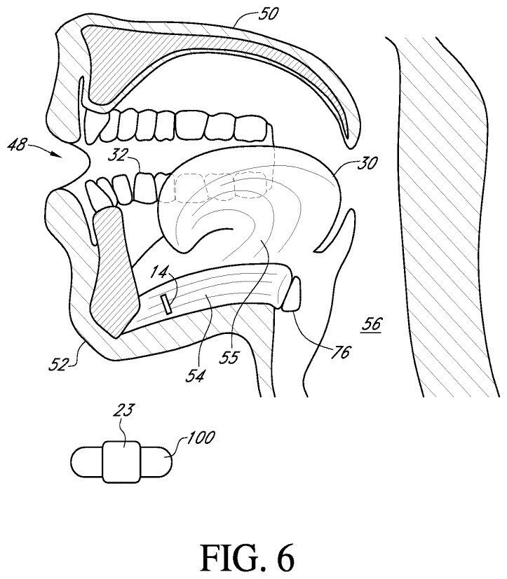

Referring to FIGS. 6-8, another embodiment of a corrective sleep apnea device 10 of the present invention is depicted. In this embodiment, the first portion 14 and the second portion 23 of the actuator combination do not interact with each other in a magnetic fashion, instead the two portions 14 and 23 interact with each other to stimulate a muscle of the oral floor, such as the genioglossus muscle 55 or the geniohyoideus muscle 54, to contract during a sleep apnea event or during sleep onset, which in turn brings the base of the tongue 30 forward, keeping the user's oral airway and posterior nasopharyngeal space 56 open while the user sleeps. In a preferred embodiment, the two portions 14 and 23 can interact with each other to stimulate the oral muscle without requiring an implanted battery by using a focusing lead/micro-implantable pulse generator as the first portion 14 of the actuator combination.

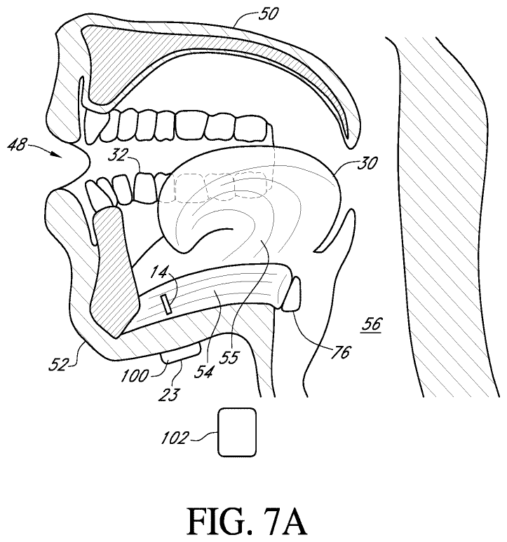

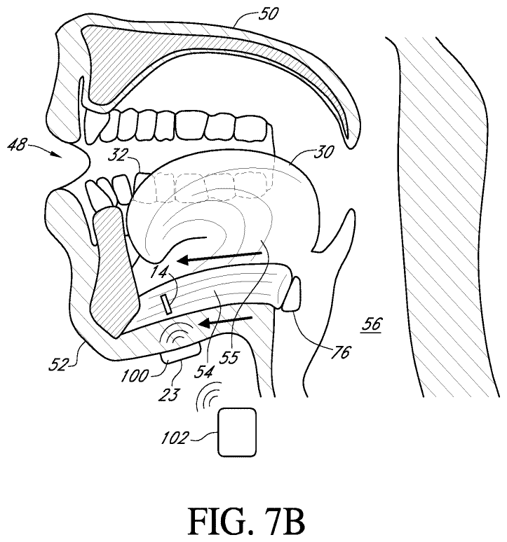

In this preferred embodiment, the first portion 14 of the actuator combination is a micro-implantable pulse generator that can stimulate the muscles of the floor of the mouth 48 or tongue 30 when triggered and does not rely on its own internal battery source. The micro-implantable pulse generator includes, among other things, an antenna and a receiver. In this embodiment, the second portion 23 of the actuator combination is housed in a disposable adhesive pad 100 and includes a control system, a battery, an antenna and a transmitter and controls the first portion 14 of the actuator combination by transmitting signals to it. Such an embodiment accommodates the endentulous patient or one that cannot utilize or tolerate a retainer. Further, in FIGS. 6 and 7A-7B, this embodiment only depicts one first portion 14 and one second portion 23 of the actuator combination being used, but it should be understood, as described in prior embodiments above, that, in various embodiments, the first portion 14 and the second portion 23 of the actuator combination can each have multiple actuators in use, and are not limited to just one.

It should be understood that in other embodiments the second portion 23 of the actuator combination can also be housed in the winglets 22a, 22b of a retainer 12, similar to the embodiments described above. In this embodiment, the first portion 14 of the actuator combination is implanted into a muscle of the oral floor, such as the genioglossus muscle 55 or the geniohyoideus muscle 54. It should be understood that the first portion 14 of the actuator combination can be implanted into a muscle of the oral floor using any of the techniques described herein (e.g. direct implantation, intraoral direct puncture, transcutaneous direct puncture, transvascular).

With first portion 14 of the actuator combination implanted as depicted in FIG. 6, the user is ready to use the corrective sleep apnea device 10 of this embodiment. In FIG. 6, the user is about to go to sleep. In this view, the user takes the disposable adhesive pad 100 and attaches it underneath his chin as depicted in FIG. 7A. The location can be identified by locating the midline between the inferior mental spine and the anterior margin of the hyoid 76. With the adhesive pad 100 in position, the second portion 23 of the actuator combination in the adhesive pad 100 now interacts with the implanted first portion 14 of the actuator combination implanted in the tongue 30, resulting in an active state of the apparatus. In a constant active state, as depicted in FIG. 7B, the second portion 23 of the actuator combination in the adhesive pad 100 transmits a signal to the implanted first portion 14 of the actuator combination to stimulate the oral muscle it is implanted within. This stimulation of the muscle of the oral floor, such as the genioglossus muscle 55 or the geniohyoideus muscle 54, causes that muscle to contract, which in turn brings the base of the tongue 30 forward, keeping the user's oral airway and posterior nasopharyngeal space 56 open while the user sleeps. In a preferred embodiment, a device 102, such as a smart phone, detects when a sleep apnea event or sleep onset is occurring and triggers the second portion 23 of the actuator combination in the adhesive pad 100 to transmit a signal to the implanted first portion 14 of the actuator combination to stimulate the connected oral muscle. In this embodiment, the actuator combination 14, 23 is not in a perpetual active state, and the implanted first portion 14 of the actuator combination is only triggered as necessary, when sleep function thresholds are reached.

In the morning, when the user is no longer sleeping, he removes the disposable adhesive pad 100 from under his chin, and the actuator portions 14, 23 are no longer in proximity, returning the actuator combination to an inactive state. The user, with the adhesive pad 100 removed, proceeds with normal daily activities.

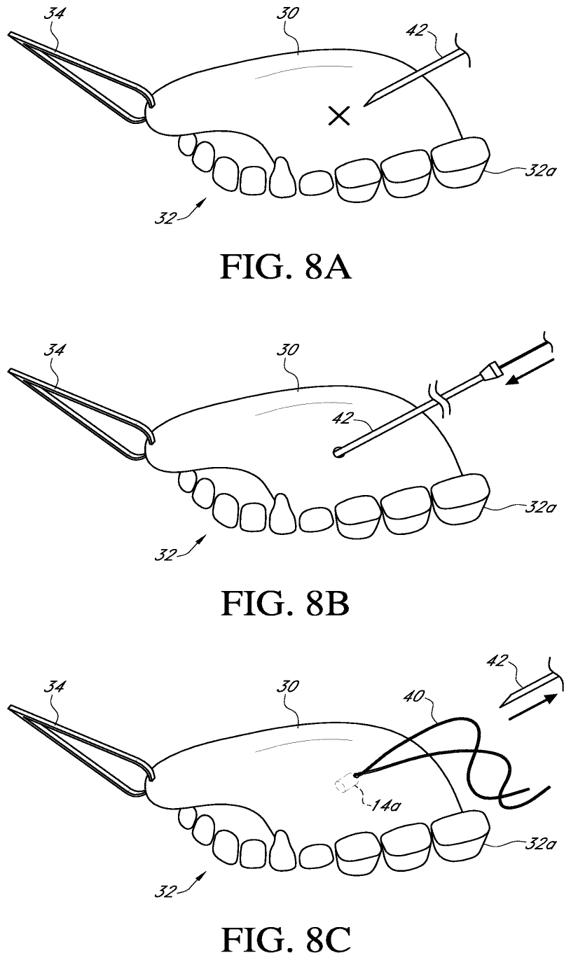

In addition to the methods described above, the first portion 14 of the actuator combination can be implanted into a user of the corrective sleep apnea device 10 using several known minimally invasive techniques. FIGS. 8A-8C depict one method for implanting the actuators 14a, 14b of the first portion 14 of the actuator combination into a user's tongue 30 using the known minimally invasive technique of an intraoral direct puncture. Referring to FIG. 8A, the person inserting the left side actuator 14a of the first portion 14 of the actuator combination uses a puller 34 to distract and pull the tongue of the user of the invention forward toward the front of the mouth. With the tongue in a forward position, the person inserting the left side actuator 14a of the first portion 14 of the actuator combination inserts a needle 42 into the tongue 30. In this embodiment, the needle 42 is inserted approximately 5 mm below the top surface of the tongue 30. In this embodiment, the tip of the needle 42 is inserted at a location such that when the left side actuator 14a of the first portion 14 of the actuator combination is implanted, it is at an optimal location to interact with the winglet 22a of a retainer 12 when the retainer 12 is in the user's mouth 48. Referring to FIG. 8B, with the tip of the needle 42 in place, the left side actuator 14a of the first portion 14 of the actuator combination is pushed through the lumen of the needle 42 and inserted into the tongue 30. After the left side actuator 14a of the first portion 14 of the actuator combination is implanted, as depicted in FIG. 8C, the area where the left side actuator 14a of the first portion 14 of the actuator combination was inserted is sutured closed, using sutures 40 with known surgical techniques. In such an embodiment, the first portion 14 of the actuator combination may be a magnet, a focusing lead/micro-implantable pulse generator or some other actuating device.

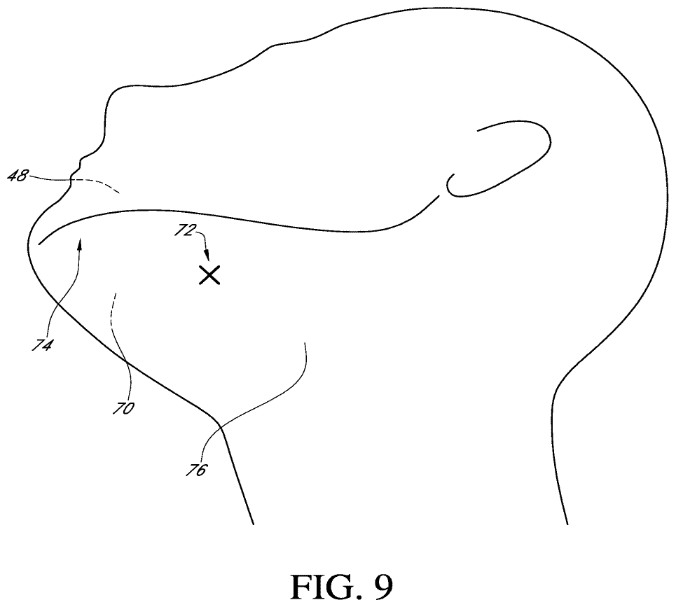

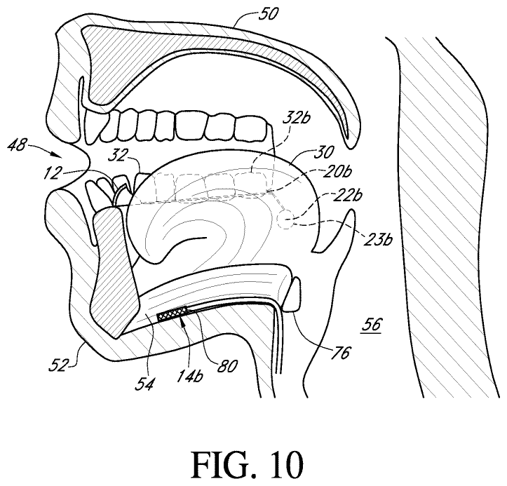

Referring to FIGS. 9 and 10, two further embodiments for implanting a first portion 14 of the actuator combination into a user's body are depicted. In FIG. 9, the first portion 14 of the actuator combination is implanted into a user's oral muscles using the known minimally invasive method of a transcutaneous direct puncture implant method. With the direct puncture implant method, in a preferred embodiment, the first portion 14 of the actuator combination is preferably cylindrical in shape, 1-3 mm in diameter with a height of approximately 3-5 mm. In this method, the person inserting the first portion 14 of the actuator combination identifies the floor 70 of the user's mouth 48. The person inserting the first portion 14 of the actuator combination inserts a needle into the floor 70 of the mouth 48. In this embodiment, the point of insertion 72 is identified by using the distance halfway between the inferior mental spine 74 and the anterior margin of the hyoid bone 76. The insertion point 72 is identified as 1 cm off midline in either direction. It should be understood that in varying embodiments, the first portion 14 of the actuator combination can be a single actuator or more than two actuators. When a single actuator is desired, the direct puncture is made in the midline. In the embodiment depicted in FIG. 9, using two actuators 14a, 14b for the first portion 14 of the actuator combination, the tip of the needle is inserted at a location such that when the left side actuator 14a of the first portion 14 of the actuator combination is implanted on the user's left side, it is at an optimal location to interact with the second portion 23 of the actuator combination when the two portions 14, 23 (e.g., their components 14, 14b, 23a, 23b) are in proximity to one another and when the actuator combination is placed in an active state. In one embodiment, the needle is similar to a Sprotte needle, with a continuous sharp portion being composed of both an inner stylet and an outer stylet. The inner stylet in an embodiment can function as an electromyographic (“EMG”) needle and identify with lying within the genioglossus muscle 55 or geniohyoideus muscle 54. When EMG confirms proper positioning, the inner stylet can be removed. In this embodiment, with the tip of the outer needle stylet in place, the first portion 14 of the actuator combination is pushed through the lumen of the needle by a pusher wire to which the actuator portion 14 is attached. The actuator portion 14 is thus inserted into the genioglossus muscle 55 or geniohyoideus muscle 54, in the pocket created by the inner stylet. After the first portion 14 of the actuator combination is implanted, it is detached from the pusher wire either mechanically or electrolytically. In such an embodiment, the first portion 14 of the actuator combination may be a magnet, a focusing lead/micro-implantable pulse generator or some other actuating device.

FIG. 10 depicts another method for implanting the first portion 14 of the actuator combination near a user's oral floor muscles using a known minimally invasive transvascular method. With the transvascular method, in a preferred embodiment, the first portion 14 of the actuator combination is preferably stent-shaped, or a hollow cylinder with the borders being a mesh or lattice configuration, 2-5 mm in diameter with a length of approximately 2-5 mm. Referring to FIG. 10, in a preferred embodiment, the person inserting the first portion 14 of the actuator combination accesses the lumen of the common femoral vein, preferably on the right side using a needle and syringe and the Seldinger technique. Access to the venous lumen is maintained by advancing a wire into the vein and exchanging the access needle and catheter for a longer specifically designed catheter. The access wire is then removed and replaced by a specifically designed wire and together with the catheter and using fluoroscopic X-ray energy, allows the person implanting the first portion 14 of the actuator combination to select the veins of the user in sequence. In this embodiment, the tip of the catheter is inserted at a location such that when the first portion 14 of the actuator combination is implanted, it is at an optimal location to interact with the second portion 23 of the actuator combination when the two portions 14, 23 are in proximity to one another or when the actuator combination is placed in an active state. In this embodiment, the first portion 14 of the actuator combination is optimally mounted to a vascular stent 80 which is preferably made of metallic alloy with shape memory such as nickel-titanium and has a cylindrical shape much like a vascular stent with the first portion 14 of the actuator combination lying within the tines, either soldered or similarly attached or otherwise embedded. Next, the wire is removed and the stent-mounted actuator 14, while connected to a pusher wire, is advanced into the catheter. The stent-mounted actuator 14 is unsheathed by holding immobile the pusher wire and pulling back on the catheter. When not constrained by the catheter, the stent-mounted actuator 14 assumes its shape, which is sized and matched to the vessel diameter by the person inserting the actuator portion 14. After the stent-mounted actuator 14 is implanted, it is detached mechanically or electrolytically. The catheter is, then, removed and the venous access site is closed in the usual fashion. In such an embodiment, the first portion 14 of the actuator combination may be a magnet, a focusing lead/micro-implantable pulse generator or some other actuating device.

Although certain embodiments and features of a corrective sleep apnea device have been described herein, the scope of coverage of this patent is not limited thereto. On the contrary, this patent covers all embodiments of the teachings of the disclosure that fairly fall within the scope of permissible equivalents.

What is claimed is:

1. A corrective sleep apnea device, comprising:
an actuator combination having a first portion and a second portion that are physically separate from each other, wherein:
the first portion is implanted into a head of a user; and
the first portion and the second portion create an active state of the actuator combination when the first portion and the second portion are in proximity to one another and when in an active state, the actuator combination moves the tongue of the user forward, keeping the oral airway and posterior nasopharyngeal space of the user open while the user sleeps.

2. The corrective sleep apnea device of claim 1, wherein the first portion is implanted into the tongue in a mouth of the head of the user.

3. The corrective sleep apnea device of claim 2, wherein the first portion is implanted into the tongue in the mouth of the user using a blade to surgically implant the first portion.

4. The corrective sleep apnea device of claim 2, wherein the first portion is implanted into the tongue in the mouth of the user using intraoral direct puncture.

5. The corrective sleep apnea device of claim 1, wherein the first portion is implanted into a mouth of the head of the user using transcutaneous direct puncture.

6. The corrective sleep apnea device of claim 1, wherein the first portion is transvascularly implanted into a vein of the head of the user.

7. A corrective sleep apnea device, comprising:
an occlusal appliance; and
an actuator combination having a first magnetic portion and a second magnetic portion that are physically separate from each other, wherein:
the first magnetic portion is implanted into a tongue of a user;
the second magnetic portion is housed in the occlusal appliance; and
the first magnetic portion and the second magnetic portion create an active state of the actuator combination when the occlusal appliance is inserted into the mouth, putting the first magnetic portion and the second magnetic portion in proximity to one another and when in an active state, the actuator combination moves the tongue of the user forward, keeping the oral airway and posterior nasopharyngeal space of the user open while the user sleeps.

8. The corrective sleep apnea device of claim 7, wherein the occlusal appliance is a retainer.

9. The corrective sleep apnea device of claim 8, wherein:
the retainer includes a plurality of winglets; and
the second magnetic portion is housed in the plurality of winglets.

10. The corrective sleep apnea device of claim 7, wherein:
the first magnetic portion includes a plurality of actuators; and
the second magnetic portion includes a plurality of actuators that each correspond with each first magnetic portion actuator.

11. The corrective sleep apnea device of claim 10, wherein the plurality of first magnetic portion actuators are discoid in shape, approximately 5 millimeters in diameter and approximately 5 millimeters in diameter.

12. The corrective sleep apnea device of claim 7, wherein the second magnetic portion is a magnetic field generator which has an active and passive state.

13. The corrective sleep apnea device of claim 12, further comprising a smart device, wherein:
the smart device is programmed with a sleep function threshold;
and when the sleep function threshold is reached, the smart device triggers the second magnetic portion to put the second magnetic portion in an active state.

14. A corrective sleep apnea device, comprising:
an adhesive pad; and
an actuator combination having a first micro-implantable pulse generator portion and a second controller portion that are physically separate from each other, wherein:
the first micro-implantable pulse generator portion is implanted into an oral muscle of a user;
the second controller portion is housed in the adhesive pad,
the first micro-implantable pulse generator portion and the second controller portion create an active state of the actuator combination when the adhesive pad is attached to the chin of the user, putting the first micro-implantable pulse generator portion and the second controller portion in proximity to one another; and
when in an active state, the actuator combination stimulates the oral muscle of the user, causing the tongue of the user to move forward, keeping the oral airway and posterior nasopharyngeal space of the user open while the user sleeps.

15. The corrective sleep apnea device of claim 14, wherein the adhesive pad is disposable.

16. The corrective sleep apnea device of claim 14, wherein:
The first micro-implantable pulse generator portion includes a plurality of actuators; and
the second controller portion includes a plurality of actuators that each correspond with each first micro-implantable pulse generator portion actuator.

17. The corrective sleep apnea device of claim 14, wherein the second controller portion has an active and passive state.

18. The corrective sleep apnea device of claim 17, further comprising a smart device, wherein:
the smart device is programmed to detect a sleep apnea event; and
when the sleep apnea event is detected, the smart device triggers the second controller portion to put the actuator combination into an active state.

19. The corrective sleep apnea device of claim 18, wherein when the sleep apnea event is no longer detected, the smart device signals the second controller portion to put the actuator combination into an inactive state.

20. The corrective sleep apnea device of claim 14, wherein the oral muscle that the first micro-implantable pulse generator portion is implanted into is the geniohyoideus muscle.
